(19) United States
(12) Patent Application Publication
(10) Pub. No.: US 2006/0142273 A1
(43) Pub. Date: Jun. 29, 2006

(54) Use of selected CGRP-antagonists in combination with other antimigraine drugs for the treatment of migraine

(75) Inventors: Klaus RUDOLF, Warthausen (DE); Henri DOODS, Warthausen (DE); Stephan Georg MUELLER, Warthausen (DE); Annette ZAMPONI, Biberach (DE); Philipp LUSTENBERGER, Warthausen (DE); Kirsten ARNDT, Biberach (DE); Gerhard SCHAENZLE, Biberach-Mettenberg (DE); Dirk STENKAMP, Biberach (DE); Rolf-Stefan Brickl, Warthausen (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International Gmbh, Ingelheim (DE)

(21) Appl. No.: 11/275,169

(22) Filed: Dec. 16, 2005

(30) Foreign Application Priority Data
Dec. 29, 2004 (DE) 102004063753

(51) Int. Cl. A61K 31/5513

(52) U.S. Cl. 514/221

(57) ABSTRACT

The present invention relates to a process for the treatment or prevention of indications which are selected from among the group comprising headaches, migraine and cluster headaches, this process comprising the joint administration of a therapeutically effective amount of a selected CGRP antagonist (A), a physiologically acceptable salt thereof or a hydrate of the salt and a therapeutically effective amount of a second or third active anti-migraine medicament (B), particularly sumatriptan, zolmitriptan or dihydroergotamine or a physiologically acceptable salt thereof, and to the corresponding pharmaceutical compositions and the preparation thereof.

### BACKGROUND TO THE INVENTION

Migraine is one of the most common neurological disorders and comprises periodic attacks of headache and nausea and a variety of other symptoms. Although considerable progress has been made, the pathophysiology of migraine is far from understood. A number of observations have, however, pointed to the involvement of the “calcitonin gene related peptide” (CGRP). Migraine headaches involve the activation of the trigeminal system and the dilation of cranial blood vessels. CGRP is located in the neurons in trigeminal ganglia, and the CGRP levels are raised during a migraine attack, which is presumably what causes the vasodilatation observed. It is therefore conceivable that inhibiting the dilation of the cranial blood vessels caused by CGRP might possibly give rise to a new treatment for migraine headaches.

Medicaments widely used for treating migraine are the so-called “triptans”, e.g. sumatriptan and zolmitriptan. These compounds derive their activity against migraine from their vasoconstrictor properties and presumably their inhibition of the release of the neuropeptide calcitonin gene related peptide (CGRP) (Ferrari, M. D., Saxena, P. R. (1995), 5-HTI receptors in migraine pathophysiology and treatment, Eur. J. Neurology, 2, 5-21; Johnson, K. W., Phebus, L. A., Cohen, M. L. (1998), Serotonin in migraine: Theories, animal models and emerging therapies, Progress in Drug Research, vol. 51, 220-244), assuming that the levels thereof are raised during a migraine attack (Edvinsson, L., Goadsby, P. J. (1994), Neuropeptides in migraine and cluster headache, Cephalgia, 14(5), 320-327). A completely new approach for the treatment of migraine is the use of CGRP antagonists (Doods, H., Hallermayer, G., Wu, D., Entzeroth, M., Rudolf, K., Engel, W., Eberlein, W. (2000), Pharmacological profile of BIBN4096BS, the first selective small molecule CGRP antagonist, Br. J. Pharmacol., 129, 420-423).

### SUMMARY OF THE INVENTION

Surprisingly it has been found that in a model assumed to predict the anti-migraine activities of pharmaceutical compositions, the combination of two or three pharmaceutical compositions with completely different modes of activity, namely a CGRP-antagonist (A) selected from among
(1) 4-(2-oxo-1,2,4,5-tetrahydro-1,3-benzodiazepin-3-yl)-piperidine-1 -carboxylic acid-{(R)-1-(4-amino-3-chloro-5-ethyl-benzyl)-2-[4-(4-methyl-piperazin-1-yl)-piperidin-1-yl ]-2-oxo-ethyl}-amide, 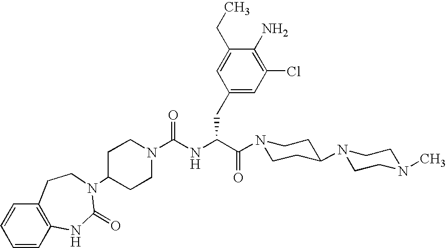
(2) [1′-((R)-3-(4-amino-3-chloro-5-trifluoromethyl-phenyl)-2-{[4-(2-oxo-1,2,4,5-tetrahydro-1,3-benzodiazepin-3-yl) -piperidine-1-carbonyl]-amino}-propionyl)-4,4′-bipiperidinyl-1-yl]-acetic acid, 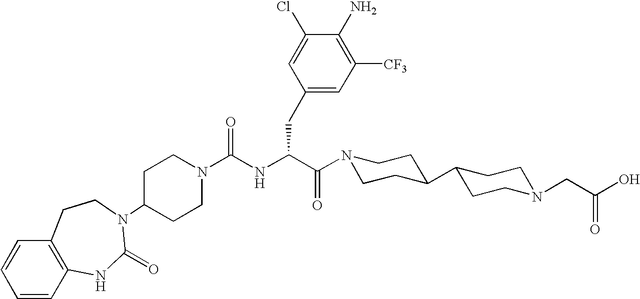
(3) 3-{1-[(R)-1-(4-amino-3,5-dibromo-benzyl)-2-[1,4′]bipiperidinyl-1′-yl-2-oxo-ethylcarbamoyl]-piperidin-4-yl}-2-oxo-1,2,3,4-tetrahydro-quinazoline-7-carboxylic acid, 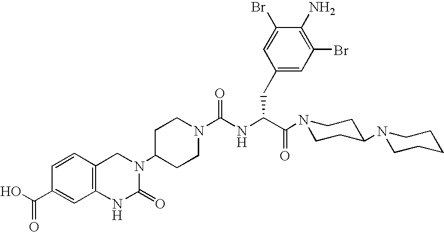
(4) (R)-1-(7-methyl-1H-benzotriazol-5-ylmethyl)-2-[4-(4-methyl-piperazin-1-yl) -piperidin-1-yl]-2-oxo-ethyl4-(2-oxo-1,2,4,5-tetrahydro-1,3-benzodiazepin-3-yl) -piperidine-1-carboxylate, 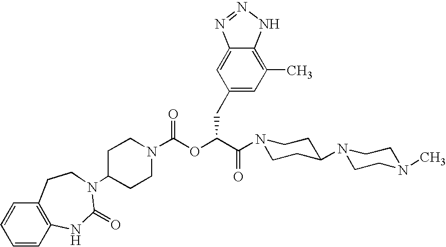
(5) (S)-2-(3-chloro-4-hydroxy-5-trifluoromethyl-benzyl)-1-[4-(1-methyl-piperidin-4-yl) -piperazin-1-yl]-4-[4-(2-oxo-1,2,4,5-tetrahydro-1,3-benzodiazepin-3-yl) -piperidin-1-yl]-butane-1,4-dione, 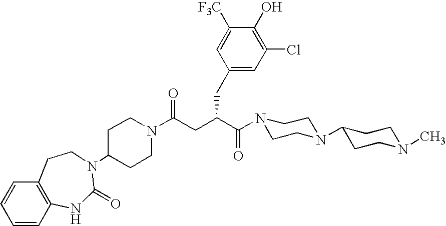
(6) (R)-1-(4-hydroxy-3,5-dimethyl-benzyl)-2-oxo-2-(4-piperidin-4-yl-piperazin-1-yl) -ethyl4-(2-oxo-1,2,4,5-tetrahydro-1,3-benzodiazepin-3-yl)-piperidine-1-carboxylate, 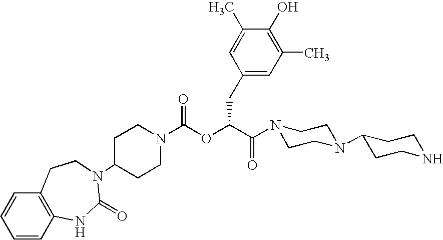
(7) (R)-1-(3,5-dibromo-4-hydroxy-benzyl)-2-[4-(1-methyl-piperidin-4-yl)-piperazin-1-yl]-2-oxo-ethyl4-(2-oxo-1,2,4,5-tetrahydro-1,3-benzodiazepin-3-yl) -piperidine-1-carboxylate, 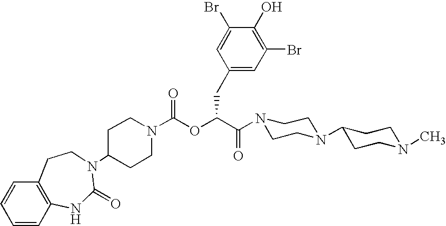
(8) (R)-1-(6-amino-5-methyl-pyridin-3-ylmethyl)-2-oxo-2-(4-piperazin-1-yl-piperidin-1-yl) -ethyl4-(2-oxo-1,2,4,5-tetrahydro-1,3-benzodiazepin-3-yl)-piperidine-1-carboxylic acid, 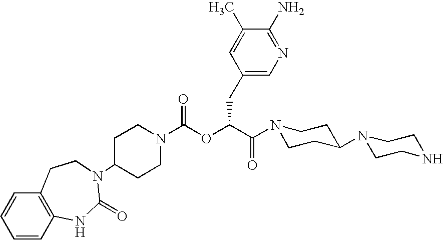
(9) (R)-1-(3,5-dibromo-4-hydroxy-benzyl)-2-oxo-2-(4-piperazin-1-yl-piperidin-1-yl) -ethyl4-(2-oxo-1,2,4,5-tetrahydro-1,3-benzodiazepin-3-yl)-piperidine-1-carboxylate, 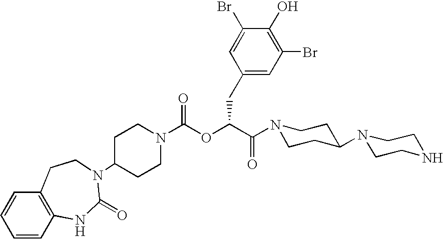
(10) (R)-1-(3,5-dibromo-4-hydroxy-benzyl)-2-oxo-2-(4-piperidin-4-yl-piperazin-1-yl) -ethyl4-(2-oxo-1,2,4,5-tetrahydro-1,3-benzodiazepin-3-yl)-piperidine-1-carboxylate, 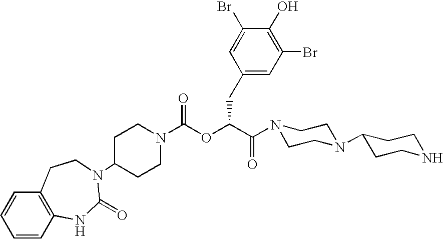
(11) (S)-2-(4-amino-3-chloro-5-trifluoromethyl-benzyl )-1-[4-(4-methyl-piperazin-1-yl) -piperidin-1-yl]-4-[4-(2-oxo-1,2,4,5-tetrahydro-1,3-benzodiazepin-3-yl) -piperidin-1-yl]-butane-1,4-dione, 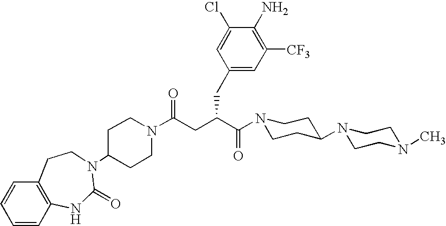
(12) 4-(2-oxo-1,2,4,5-tetrahydro-1,3-benzodiazepin-3-yl)-piperidine-1-carboxylic acid-{(R)-1-(3,4-diethyl-benzyl)-2-[4-(1-methyl-piperidin-4-yl)-piperazin-1-yl]-2-oxo-ethyl}-amide, 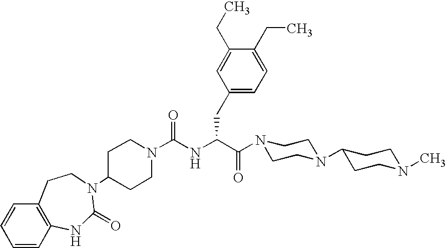
(13) (R)-1-(4-amino-3-chloro-5-trifluoromethyl-benzyl)-2-[4-(4-methyl-piperazin-1-yl) -piperidin-1-yl]-2-oxo-ethyl 4-(2-oxo-1,2,4,5-tetrahydro-1,3-benzodiazepin-3-yl) -piperidine-1-carboxylate, 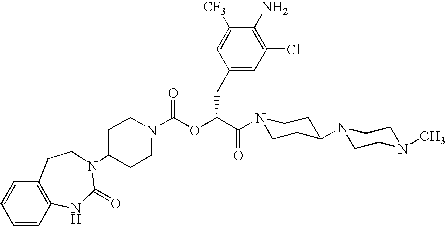
(14) 4-(2-oxo-1,2,4,5-tetrahydro-1,3-benzodiazepin-3-yl)-piperidine-1-carboxylic acid-{(R)-1-(4-amino-3-chloro-5-trifluoromethyl-benzyl)-2-[4-(4-methyl-piperazin-1-yl) -piperidin-1-yl]-2-oxo-ethyl}-amide, 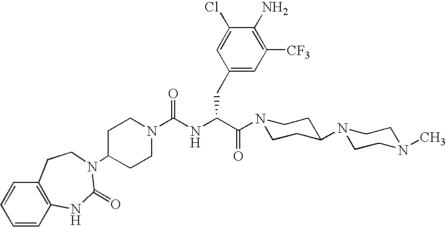
(15) (S)-2-(4-amino-3,5-bis-trifluoromethyl-benzyl)-1-[4-(4-methyl-piperazin-1-yl)-piperidin-1-yl]-4-[4-(2-oxo-1,2,4,5-tetrahydro-1,3-benzodiazepin-3-yl) -piperidin-1-yl]-butane-1,4-dione, 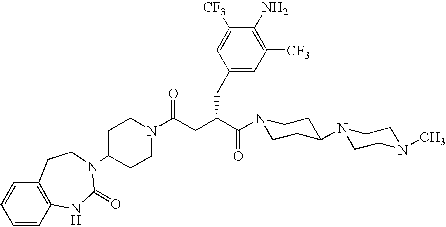
(16) 4-(2-oxo-1,2,4,5-tetrahydro-1,3-benzodiazepin-3-yl)-piperidine-1-carboxylic acid-{(R)-1-(4-amino-3,5-bis-trifluoromethyl-benzyl)-2-[4-(4-methyl-piperazin-1-yl) -piperidin-1-yl]-2-oxo-ethyl}-amide, 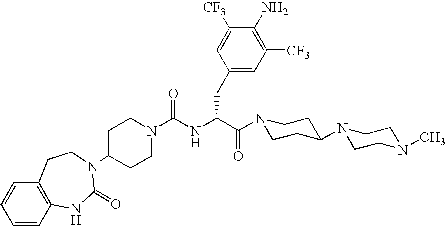
(17) (R)-1-(4-amino-3,5-bis-trifluoromethyl-benzyl)-2-[4-(4-methyl-piperazin-1-yl) -piperidin-1-yl]-2-oxo-ethyl4-(2-oxo-1,2,4,5-tetrahydro- 1,3-benzodiazepin-3-yl) -piperidine-1-carboxylate, 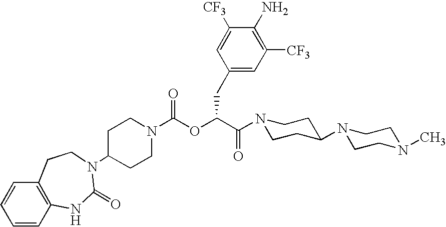
(18) 4-(2-oxo-1,2,4,5-tetrahydro-1,3-benzodiazepin-3-yl)-piperidine-1-carboxylic acid-{(R)-1-(4-amino-3-chloro-5-methyl-benzyl)-2-[4-(1-methyl-piperidin-4-yl) -piperazin-1-yl]-2-oxo-ethyl}-amide, 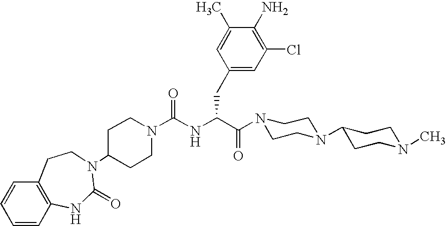
(19) (R)-1-(3,5-dibromo-4-hydroxy-benzyl)-2-[4-(4-methyl-piperazin-1-yl)-piperidin-1-yl]-2-oxo-ethyl4-(2-oxo-1,2,4,5-tetrahydro-1,3-benzodiazepin-3-yl) -piperidine-1-carboxylate, 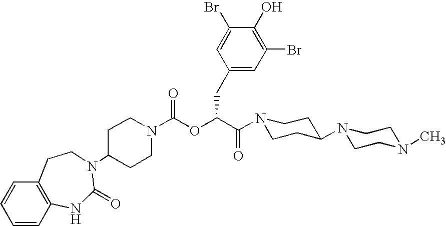
(20) (R)-1-(4-hydroxy-3,5-dimethyl-benzyl)-2-oxo-2-(4-piperazin-1-yl-piperidin-1-yl)-ethyl 4-(2-oxo-1,2,4,5-tetrahydro-1,3-benzodiazepin-3-yl)-piperidine-1-carboxylate, 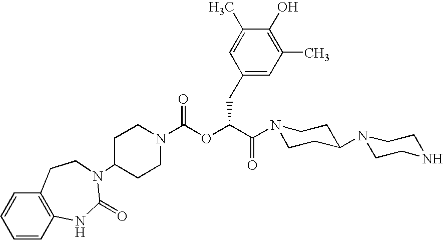
(21) (R)-1-(4-hydroxy-3,5-dimethyl-benzyl)-2-[4-(1-methyl-piperidin-4-yl)-piperazin-1-yl]-2-oxo-ethyl 4-(2-oxo-1,2,4,5-tetrahydro-1,3-benzodiazepin-3-yl) -piperidine-1-carboxylate, 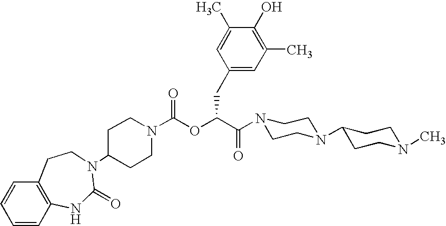
(22) (S)-1-1,4′-bipiperidinyl-1′-yl-2-(3-chloro-4-hydroxy-5-trifluoromethyl-benzyl)-4-[4-(2-oxo-1,2,4,5-tetrahydro-1,3-benzodiazepin-3-yl)-piperidin-1-yl]-butane-1,4-dione, 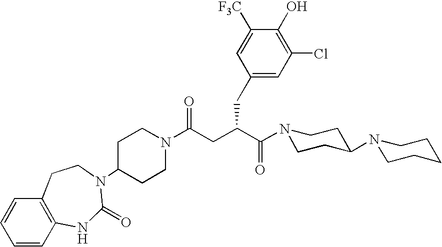
   the physiologically acceptable salts thereof and the hydrates of the salts and a 5-HT_{1B/1D}-agonist or an ergot alkaloid (B) leads to an improved activity compared with the activity of only one medicament.

### DETAILED DESCRIPTION OF THE INVENTION

In a first aspect the present invention relates to a process for the treatment or prevention of indications which are selected from among the group comprising headaches, migraine and cluster headaches, this process comprising the joint administration of a therapeutically effective amount of one of the selected CGRP-antagonists (A) according to the invention or a physiologically acceptable salt thereof or a hydrate of the salt and a therapeutically effective amount of a second or third active anti-migraine medicament (B) to a person in need of such treatment. The combination with two other active medicaments is useful for example when a CGRP antagonist (A) combined with a medicament (B) has a synergistic effect against pain, but at the same time an antiemetic activity is also desired.

The medicament (B) may be selected from among the angiotensin-II antagonists, α-agonists and α-antagonists, 5-HT_{1B/1D}-agonists, AMPA antagonists, antidepressants, antiemetics, anticonvulsants, antimuscarinics, β-blockers, calcium antagonists, corticosteroids, ergot alkaloids, histamine-H1-receptor antagonists, weak analgesics, neurokinin antagonists, neuroleptics, non-steroidal antiinflammatories, NO-synthase inhibitors, prokinetics and serotonin-reuptake inhibitors.

A non-steroidal antiinflammatory may be selected from among acclofenac, acemetacin, acetylsalicylic acid, acetaminophene (paracetamol), azathioprine, diclofenac, diflunisal, fenbufen, fenoprofen, flurbiprofen, ibuprofen, indometacin, ketoprofen, leflunomide, lornoxicam, mefenamic acid, naproxen, phenylbutazone, piroxicam, sulphasalazine, tenoxicam, zomepirac and the physiologically acceptable salts thereof, meloxicam and other selective COX2-inhibitors, such as for example celecoxib, etoricoxib, parecoxib, rofecoxib and valdecoxib, as well as substances which inhibit the earlier or later stages of prostaglandin synthesis, or prostaglandin receptor antagonists, such as for example EP2-receptor antagonists and IP-receptor antagonists.

Examples of angiotensin-II antagonists which may be used are described in EP-A-253310, EP-A-323841, EP-A-324377, EP-A-420237, EP-A-43983, EP-A-459136, EP-A-475206, EP-A-502314, EP-A-504888, EP-A-514198, WO 91/14679, WO 93/20816, U.S. Pat. No. 4,355,040 and U.S. Pat. No. 4,880,804, or the physiologically acceptable salts thereof. Preferred angiotensin II antagonists are sartans, such as candesartan, eprosartan, irbesartan, losartan, olmesartan, tasosartan, telmisartan or valsartan.

Examples of 5-HT_{1B/1D}-agonists which may be used are almotriptan, avitriptan, donitriptan, eletriptan, frovatriptan, naratriptan, rizatriptan, sumatriptan or zolmitriptan or the physiologically acceptable salts thereof.

Suitable ergot alkaloids include e.g. ergotamine and dihydroergotamine; and examples of serotonin reuptake inhibitors which may be used are citalopram, duloxetine, escitalopram, fluoxetine, fluvoxamine, paroxetine, sertraline, trazodone or the physiologically acceptable salts thereof.

Additional active substances which may be considered for use as component (B) in the above-mentioned combinations include e.g. metoclopramide, domperidone, diphenhydramine, cyclizine, promethazine, chlorpromazine, vigabatrin, timolol, isomethepten, pizotifen, botox, gabapentin, pregabalin, topiramat, riboflavin, montelukast, lisinopril, micardis, prochlorperazine, dexamethasone, flunarizine, dextropropoxyphen, meperidin, metoprolol, propranolol, nadolol, atenolol, clonidine, indoramine, carbamazepine, phenytoin, valproate, amitryptiline, imipramine, venlafaxine, lidocaine or diltiazem.

According to a preferred embodiment of the process according to the invention medicament (B) is selected from among the ergot alkaloids and 5-HT_{1B/1D}-agonists, while dihydroergotamine, sumatriptan and zolmitriptan are particularly preferred according to the invention and sumatriptan or the physiologically acceptable salts thereof are most preferred.

According to another preferred embodiment of the process according to the invention medicament (B) is selected from among the non-steroidal antiinflammatories, of which meloxicam or the physiologically acceptable salts thereof are particularly preferred.

According to another preferred embodiment of the process according to the invention medicament (B) is selected from among the serotonin reuptake inhibitors, of which duloxetine or the physiologically acceptable salts thereof are particularly preferred.

The dosage for the combined migraine drug (B) is roughly 1/50 of the lowest normally recommended dose to 1/1 of the normally recommended dose, by oral, nasal, inhalative, subcutaneous or intravenous route. The normally recommended dose for the combined migraine drug (B) is deemed to be the dose specified in the Rote Liste Win^{R} 2001/I, Editio Cantor Verlag Aulendorf.

According to the invention the selected CGRP antagonists (A) or a physiologically acceptable salt thereof or a hydrate of the salt may be administered by intravenous or subcutaneous route in a dosage of 0.0001 to 3 mg/kg of body weight, by oral route in a dosage of 0.1 to 20 mg/kg body weight or by nasal or inhalative route in a dosage of 0.1 to 10 mg/kg body weight once, twice or three times a day, in combination with
sumatriptan or a physiologically acceptable salt thereof, which may be administered by oral route in a dosage of 0.03 to 1.43 mg/kg body weight once, twice or three times a day or
by intravenous or subcutaneous route in a dosage of 0.002 to 0.09 mg/kg body weight once or twice a day or
by rectal route in a dosage of 0.007 to 0.36 mg/kg body weight once or twice a day or
by nasal route in a dosage of 0.006 to 0.29 mg/kg body weight once or twice a day, or combined with
zolmitriptan or a physiologically acceptable salt thereof, which may be administered by oral route in a dosage of 0.0007 to 0.036 mg/kg body weight once or twice a day, or
combined with dihydroergotamine or a physiologically acceptable salt thereof, which may be administered by oral route in a dosage of 0.001 to 0.07 mg/kg body weight once or twice a day, or
combined with meloxicam or a physiologically acceptable salt thereof, which may be administered by oral route in a dosage of 0.004 to 0.21 mg/kg body weight once a day or
combined with duloxetine or a physiologically acceptable salt thereof, which may be administered by oral route in a dosage of 0.03 to 1.43 mg/kg body weight once, twice or three times a day or
by intravenous or subcutaneous route in a dosage of 0.002 to 0.09 mg/kg body weight once or twice a day or
by rectal route in a dosage of 0.007 to 0.36 mg/kg body weight once or twice a day or
by nasal route in a dosage of 0.006 to 0.29 mg/kg body weight once or twice a day.

In a second aspect the present invention provides a pharmaceutical composition for the treatment or prevention of headaches, migraine or cluster headache, which consists of a therapeutically effective amount of a selected CGRP-antagonist (A), a physiologically acceptable salt thereof or a hydrate of the salt and an anti-migraine medicament (B), selected from among sumatriptan, zolmitriptan and dihydroergotamine or a physiologically acceptable salt thereof, as a combined preparation for simultaneous or sequential administration.

A pharmaceutical composition according to the invention may contain a single dosage unit of 0.1 to 1500 mg, preferably 0.3 to 1000 mg, particularly preferably 5 to 750 mg, of a selected CGRP-antagonist (A), an equivalent amount of a physiologically acceptable salt thereof or a hydrate of the salt and
a single dosage unit of 1 to 100 mg sumatriptan or
a single dosage unit of 0.1 to 2.5 mg zolmitriptan or
a single dosage unit of 0.1 to 5 mg dihydroergotamine or
a single dosage unit of 7.5 to 15 mg meloxicam or
a single dosage unit of 0.1 to 150 mg, preferably 0.2 to 100 mg, for example 10 to 100 mg, particularly preferably 10 to 80 mg, particularly 40 to 80 mg, of duloxetine.

All the doses or dosage units of a physiologically acceptable salt of one of the above-mentioned active compounds should be understood as being doses or dosages of the active compound itself.

Moreover a pharmaceutical composition according to the invention may be a kit of parts for the treatment or prevention of headache, migraine or cluster headaches, the kit comprising:
(a) a first enclosure containing a pharmaceutical composition comprising a therapeutically effective amount of a selected CGRP antagonists (A), a physiologically acceptable salt thereof or a hydrate of the salt and one or more physiologically acceptable diluents and/or carriers; and
(b) a second enclosure containing a pharmaceutical composition comprising sumatriptan, zolmitriptan or dihydroergotamine or a physiologically acceptable salt thereof and one or more physiologically acceptable diluents and/or carriers.

A preferred kit of parts comprises sumatriptan in the second enclosure.

In a third aspect the present invention relates to the use of a selected CGRP antagonist (A), a physiologically acceptable salt thereof or a hydrate of the salt combined with a second or third anti-migraine medicament (B) for preparing a pharmaceutical composition for the treatment or prevention of headaches, migraine or cluster headache. Medicament (B) and preferred embodiments thereof as well as pharmaceutical compositions are mentioned above in the first and second aspects of the invention. Most preferred of all aspects of the invention is the combination of a selected CGRP antagonist (A), a physiologically acceptable salt thereof or a hydrate of the salt with sumatriptan or physiologically acceptable salts thereof.

A number of the above-mentioned medicament components (B) are already on the market; e.g. sumatriptan is sold under the trade mark Imigran®, zolmitriptan is sold under the trade mark Ascotop®, meloxicam is sold under the trade mark Mobec® and dihydroergotamine and the physiologically acceptable salts thereof are sold under the trade mark Agit®.

The selected CGRP antagonists (A) may be administered in conjunction with a second or third additional anti-migraine medicament (B1 and B2) e.g. using one of the following pharmaceutical formulations.

To achieve optimum dosages and compliance, the double or triple combinations according to the invention as administered as fixed combinations in different preparations as described in the following paragraphs. As a rapid onset of activity is advantageous in treating migraine, the oral forms were adapted to obtain a rapid release of active substance. If, however, one or more components is also required to have a long-lasting effect (e.g. in the case of certain non-steroidal antiinflammatories or antiemetics, some of which have to be administered three to four times a day) in order to avoid the need for administration several times a day one or more components may also be designed to be released slowly. This may be by the use of slowly releasing matrix tablets or—preferably—by using multiparticulate systems such as pellets or extruded materials, to reduce the intra- and interindividual variability.

Preferred preparations are:
capsules for powder inhalation, containing 0.1 to 50 mg, preferably 0.3 to 30 mg, of (A) and varying amounts of other anti-migraine medicaments (B);
nasal spray containing 2 to 50 mg, preferably 5 to 40 mg, (A) and correspondingly varying amounts of other anti-migraine medicaments (B);
tablets containing 10 to 600 mg, preferably 30 to 400 mg, (A) and correspondingly varying amounts of other anti-migraine medicaments (B);
pellets for capsules, containing varying parts by weight (A) and correspondingly varying amounts of other anti-migraine medicaments (B);
extruded materials for capsules or tablets, containing varying parts by weight of (A) and correspondingly varying amounts of other anti-migraine medicaments (B);
suppositories containing 10 to 600 mg, preferably 30 to 400 mg, of (A) and correspondingly varying amounts of other anti-migraine medicaments (B);
injectable solutions containing 0.2 to 30 mg, preferably 0.5 to 15 mg, of (A) and correspondingly varying amounts of other anti-migraine medicaments (B).

The following Examples describe pharmaceutical preparations which contain as active substance a selected CGRP antagonist (A) combined with one or two other medicaments active against migraine (B). Preceding them is first of all a Table in which numbers are assigned to the medicament components, to identify the active substances in the following Tables of Examples.

Medicament components

| substance | |
|---|---|
| no. | substance A, B |
| 1 | CGRP antagonist (1) or its hydrochloride-pentahydrate [1a] |
| 2 | CGRP antagonist (2) or a physiologically acceptable salt |
| | thereof [2a] |
| 3 | CGRP antagonist (3) or a physiologically acceptable salt |
| | thereof [3a] |
| 4 | CGRP antagonist (4) or a physiologically acceptable salt |
| | thereof [4a] |
| 5 | CGRP antagonist (5) or a physiologically acceptable salt |
| | thereof [5a] |
| 6 | CGRP antagonist (6) or a physiologically acceptable salt |
| | thereof [6a] |
| 7 | CGRP antagonist (7) or a physiologically acceptable salt |
| | thereof [7a] |
| 8 | CGRP antagonist (8) or a physiologically acceptable salt |
| | thereof [8a] |
| 9 | CGRP antagonist (9) or a physiologically acceptable salt |
| | thereof [9a] |
| 10 | CGRP antagonist (10) or a physiologically acceptable salt |
| | thereof [10a] |
| 11 | CGRP antagonist (11) or a physiologically acceptable salt |
| | thereof [11a] |
| 12 | CGRP antagonist (12) or a physiologically acceptable salt |
| | thereof [12a] |
| 13 | CGRP antagonist (13) or a physiologically acceptable salt |
| | thereof [13a] |
| 14 | CGRP antagonist (14) or a physiologically acceptable salt |
| | thereof [14a] |
| 15 | CGRP antagonist (15) or a physiologically acceptable salt |
| | thereof [15a] |
| 16 | CGRP antagonist (16) or a physiologically acceptable salt |
| | thereof [16a] |
| 17 | CGRP antagonist (17) or a physiologically acceptable salt |
| | thereof [17a] |
| 18 | CGRP antagonist (18) or a physiologically acceptable salt |
| | thereof [18a] |
| 19 | CGRP antagonist (19) or a physiologically acceptable salt |
| | thereof [19a] |
| 20 | CGRP antagonist (20) or a physiologically acceptable salt |
| | thereof [20a] |
| 21 | CGRP antagonist (21) or a physiologically acceptable salt |
| | thereof [21a] |
| 22 | CGRP antagonist (22) or a physiologically acceptable salt |
| | thereof [22a] |
| 23 | sumatriptan |
| 23a | sumatriptan hydrogen succinate |
| 24 | almotriptan |
| 24a | almotriptan[(RS)-hydrosuccinate] |
| 25 | eletriptan |
| 25a | eletriptan hydrobromide |
| 26 | frovatriptan |
| 26a | frovatriptan succinate |
| 27 | naratriptan |
| 27a | naratriptan hydrochloride |
| 28 | rizatriptan |
| 28a | rizatriptan benzoate |
| 29 | zolmitriptan |
| 30 | propranolol-HCl |
| 31 | aceclophenac |
| 32 | acemetacin |
| 33 | azathioprin |
| 34 | diclofenac-sodium |
| 35 | celecoxib |
| 36 | diflunisal |
| 37 | fenoprofen-calcium |
| 38 | flurbiprofen |
| 39 | ibuprofen |
| 40 | indometacin |
| 41 | ketoprofen |
| 42 | leflunomid |
| 43 | lornoxicam |
| 44 | mefenamic acid |
| 45 | meloxicam |
| 46 | naproxen |
| 47 | phenylbutazone |
| 48 | piroxicam |
| 49 | sulphasalazine |
| 50 | irbesartan |
| 51 | valsartan |
| 52 | eprosartan |
| 52a | eprosartan mesilate |
| 53 | losartan-potassium |
| 54 | olmesartan medoxomil |
| 55 | telmisartan |
| 56 | candesartan cilexetil |
| 57 | metoclopramide |
| 57a | metoclopramide-HCl 1H2O |
| 58 | domperidone |
| 58a | domperidone maleate |
| 59 | diphenhydramine |
| 60 | chlorpromazine |
| 60a | chlorpromazine-HCl |
| 61 | dexamethasone |
| 62 | flunarizine |
| 62a | flunarizine hydrochloride |
| 63 | dextroproxyphen |
| 64 | nadolol |
| 65 | atenolol |
| 66 | clonidine |
| 67 | indoramine-HCl |
| 68 | carbamazepine |
| 69 | phenytoin |
| 70 | promethazine-HCl |
| 71 | duloxetine |

### EXAMPLE 1a

### Tablets Containing 100 mg CGRP Antagonist and 50 mg Sumatriptan

Composition/tablet:

| | | |
|---|---|---|
| CGRP antagonist | 100 | mg |
| sumatriptan | 70 | mg (corresponds to 50 mg sumatriptan) |
| hydrogen succinate | | |
| lactose | 375 | mg |
| magnesium stearate | 3.0 | mg |
| povidone | 8.5 | mg |
| crospovidone | 14.4 | mg |
| volatile constituent: | | |
| water | | |

Preparation

CGRP antagonist, sumatriptan hydrogen succinate and lactose (fine) are homogeneously mixed in a suitable mixer (e.g. Diosna P2); then the mixture is granulated with an aqueous povidone solution. The granulated material is screened through a 1.6 mm Kressner screen and dried for 2 hours at 40° C. Then the granules are screened in a suitable mill, e.g. a Comill, at 3000 rpm with a mesh size of 1.1 mm. The granules are then mixed with crospovidone for 5 minutes and then with magnesium stearate for 1 minute. The mixture thus obtained is compressed in a tablet press to produce tablets of suitable diameter.

### EXAMPLE 1b

### Tablets Containing 10 mg CGRP Antagonist and 50 mg Sumatriptan

Composition/tablet:

| | | |
|---|---|---|
| CGRP antagonist | 10 | mg sumatriptan hydrogen succinate |
| | 70 | mg (corresponds to 50 mg sumatriptan) |
| lactose | 475 | mg |
| magnesium stearate | 3.0 | mg |
| povidone | 8.5 | mg |
| crospovidone | 14.4 | mg |
| volatile constituent: | | |
| water | | |

Preparation

CGRP antagonist, sumatriptan hydrogen succinate and lactose (fine) are homogeneously mixed in a suitable mixer (e.g. Diosna P2); then the mixture is granulated with an aqueous povidone solution. The granulated material is screened through a 1.6 mm Kressner screen and dried for 2 hours at 40° C. Then the granules are screened in a suitable mill, e.g. a Comill, at 3000 rpm with a mesh size of 1.1 mm. The granules are then mixed with crospovidone for 5 minutes and then with magnesium stearate for 1 minute. The mixture thus obtained is compressed in a tablet press to produce tablets of suitable diameter.

### EXAMPLE 1c

### Tablets Containing 600 mg CGRP Antagonist and 50 mg Sumatriptan

Composition/tablet:

| | | |
|---|---|---|
| CGRP antagonist | 600 | mg |
| sumatriptan hydrogen | 70 | mg (corresponds to 50 mg sumatriptan) |
| succinate | | |
| lactose | 75 | mg |
| magnesium stearate | 6 | mg |
| povidone | 17 | mg |
| crospovidone | 28.8 | mg |
| volatile constituent: | | |
| water | | |

Preparation

CGRP antagonist, sumatriptan hydrogen succinate and lactose (fine) are homogeneously mixed in a suitable mixer (e.g. Diosna P2); then the mixture is granulated with an aqueous povidone solution. The granulated material is screened through a 1.6 mm Kressner screen and dried for 2 hours at 40° C. Then the granules are screened in a suitable mill, e.g. a Comill, with a mesh size of 1.1 mm at 3000 rpm. The granules are then mixed with crospovidone for 5 minutes and then with magnesium stearate for 1 minute. The mixture thus obtained is compressed in a tablet press to produce tablets of suitable diameter.

The weight of the tablet is 911 mg.

### EXAMPLE 1d

### Tablets Containing 100 mg CGRP Antagonist, 50 mg Sumatriptan and 10 mg domperidone

Composition/tablet:

| | | |
|---|---|---|
| CGRP antagonist | 100 | mg |
| sumatriptan hydrogen | 70 | mg (corresponds to 50 mg sumatriptan) |
| succinate | | |
| domperidone maleate | 12.7 | mg (corresponds to 10 mg domperidone) |
| lactose | 403 | mg |
| magnesium stearate | 3.1 | mg |
| povidone | 9.1 | mg |
| crospovidone | 15.3 | mg |
| volatile constituent: | | |
| water | | |

Preparation

CGRP antagonist, sumatriptan hydrogen succinate, domperidone maleate and lactose (fine) are homogeneously mixed in a suitable mixer (e.g. Diosna P2); then the mixture is granulated with an aqueous povidone solution. The granulated material is screened through a 1.6 mm Kressner screen and dried for 2 hours at 40° C. Then the granules are screened in a suitable mill, e.g. a Comill, with a mesh size of 1.1 mm at 3000 rpm. The granules are then mixed with crospovidone for 5 minutes and then with magnesium stearate for 1 minute. The mixture thus obtained is compressed in a tablet press to produce tablets of suitable diameter.

This method is the basis for other examples of combinations listed in the Table that follows.

In these Examples 10 to 600 mg CGRP antagonist (A) was used, either in active form or in the form of a physiologically acceptable salt, combined with an amount, as listed in the Table, of an additional active substance (B) or one of the physiologically acceptable salts thereof.

Table relating to Example 1a-d

| | | | | | | | | | | mg of | |
|---|---|---|---|---|---|---|---|---|---|---|---|
| | Subst. | | Subst. | | Subst. | | mg of | mg of | mg of | Mg- | Ø |
| Ex. | A no. | mg | B1 no. | mg | B2 no. | mg | lactose | povidone | crospovidone | stearate | [mm] |
| 1.1 | 13 | 180 | 38 | 70 | | | 500.0 | 11.3 | 19.0 | 3.9 | 12 |
| 1.2 | 6a | 290 | 28 | 70 | 45 | 25 | 570.0 | 14.3 | 24.2 | 5.0 | 13 |
| 1.3 | 21a | 100 | 24 | 14.5 | | | 229.1 | 5.2 | 8.7 | 1.8 | 10 |
| 1.4 | 2 | 50 | 54 | 2.5 | | | 105.0 | 2.4 | 4.0 | 0.8 | 10 |
| 1.5 | 6 | 360 | 44 | 2.5 | 58 | 60 | 495.0 | 13.8 | 23.3 | 4.8 | 12 |
| 1.6 | 3 | 40 | 28 | 40 | | | 160.0 | 3.6 | 6.1 | 1.2 | 11 |
| 1.7 | 17 | 150 | 66 | 100 | | | 500.0 | 11.3 | 19.0 | 3.9 | 13 |
| 1.8 | 3a | 170 | 30 | 15 | | | 370.0 | 8.3 | 14.1 | 2.9 | 10 |
| 1.9 | 14 | 330 | 23 | 15 | 54 | 200 | 400.0 | 14.2 | 24.0 | 4.9 | 13 |
| 1.10 | 5 | 600 | 48 | 5.9 | | | 300.0 | 13.6 | 23.0 | 4.7 | 10 |
| 1.11 | 1 | 10 | 26 | 75 | | | 170.0 | 3.8 | 6.5 | 1.3 | 12 |
| 1.12 | 16 | 360 | 39 | 12.5 | | | 465.0 | 12.6 | 21.3 | 4.4 | 10 |
| 1.13 | 3 | 160 | 41 | 40 | | | 400.0 | 9.0 | 15.2 | 3.1 | 11 |
| 1.14 | 4a | 170 | 61 | 40 | 28 | 25 | 470.0 | 10.6 | 17.9 | 3.7 | 12 |
| 1.15 | 3 | 370 | 43a | 10.5 | | | 361.0 | 11.1 | 18.8 | 3.9 | 10 |
| 1.16 | 22 | 310 | 64a | 12.7 | | | 485.0 | 12.1 | 20.5 | 4.2 | 10 |
| 1.17 | 1a | 270 | 44a | 12.7 | 23 | 2.3 | 570.0 | 12.8 | 21.7 | 4.4 | 10 |
| 1.18 | 5 | 230 | 46a | 25 | | | 510.0 | 11.5 | 19.4 | 4.0 | 11 |
| 1.19 | 15a | 60 | 70 | 60 | | | 240.0 | 5.4 | 9.1 | 1.9 | 12 |
| 1.20 | 4 | 380 | 52 | 0.15 | | | 495.0 | 13.1 | 22.2 | 4.6 | 10 |
| 1.21 | 12a | 60 | 53 | 27.6 | | | 175.2 | 3.9 | 6.7 | 1.4 | 11 |
| 1.22 | 5 | 330 | 56 | 60 | | | 500.0 | 13.4 | 22.6 | 4.6 | 12 |
| 1.23 | 7 | 300 | 57 | 25 | | | 300.0 | 9.4 | 15.9 | 3.3 | 11 |
| 1.24 | 17a | 160 | 58 | 60 | | | 440.0 | 9.9 | 16.7 | 3.4 | 12 |
| 1.25 | 4 | 150 | 58 | 60 | 30 | 15 | 450.0 | 10.1 | 17.1 | 3.5 | 12 |

### EXAMPLE 2a

### Tablets Containing 100 mg CGRP Antagonist and 50 mg Sumatriptan

Composition:

| | | |
|---|---|---|
| CGRP antagonist | 100 | mg |
| sumatriptan hydrogen | 70 | mg (corresponds to 50 mg sumatriptan) |
| succinate | | |
| lactose | 284 | mg |
| microcrystalline cellulose | 89.5 | mg |
| magnesium stearate | 7.2 | mg |
| croscarmellose | 7.3 | mg |
| volatile constituent: | | |
| water | | |

Preparation

CGRP antagonist (A), sumatriptan hydrogen succinate, lactose (fine) and microcrystalline cellulose are homogeneously mixed in a suitable mixer (e.g. Diosna P2); then the mixture is granulated with water. The granulated material is screened through a 1.6 mm Kressner screen and dried for 2 hours at 40° C. Then the granules are screened in a suitable mill, e.g. a Comill, at 3000 rpm with a mesh size of 1.1 mm. The granules are then mixed with croscarmellose for 5 minutes and then with magnesium stearate for 1 minute. The mixture thus obtained is compressed in a tablet press to produce tablets of suitable diameter.

### EXAMPLE 2b

Tablets Containing 10 mg CGRP Antagonist and 50 mg Sumatriptan

Composition:

| | | |
|---|---|---|
| CGRP antagonist | 10 | mg |
| sumatriptan hydrogen | 70 | mg (corresponds to 50 mg sumatriptan) |
| succinate | | |
| lactose | 274 | mg |
| microcrystalline cellulose | 109.5 | mg |
| magnesium stearate | 7.2 | mg |
| croscarmellose | 7.3 | mg |
| volatile constituent: | | |
| water | | |

Preparation

CGRP antagonist, sumatriptan hydrogen succinate, lactose (fine) and microcrystalline cellulose are homogeneously mixed in a suitable mixer (e.g. Diosna P2); then the mixture is granulated with water. The granulated material is screened through a 1.6 mm Kressner screen and dried for 2 hours at 40° C. Then the granules are screened in a suitable mill, e.g. a Comill, with a mesh size of 1.1 mm at 3000 rpm. The granules are then mixed with croscarmellose for 5 minutes and then with magnesium stearate for 1 minute. The mixture thus obtained is compressed in a tablet press to produce tablets of suitable diameter.

### EXAMPLE 2c

### Tablets Containing 400 mg CGRP Antagonist and 50 mg Sumatriptan

Composition:

| | | |
|---|---|---|
| CGRP antagonist | 400 | mg |
| sumatriptan hydrogen | 70 | mg (corresponds to 50 mg sumatriptan) |
| succinate | | |
| lactose | 194 | mg |
| microcrystalline cellulose | 89.5 | mg |
| magnesium stearate | 7.2 | mg |
| croscarmellose | 7.3 | mg |
| volatile constituent: | | |
| water | | |

Preparation

CGRP antagonist, sumatriptan hydrogen succinate, lactose (fine) and microcrystalline cellulose are homogeneously mixed in a suitable mixer (e.g. Diosna P2); then the mixture is granulated with water. The granulated material is screened through a 1.6 mm Kressner screen and dried for 2 hours at 40° C. Then the granules are screened in a suitable mill, e.g. a Comill, with a mesh size of 1.1 mm at 3000 rpm. The granules are then mixed with croscarmellose for 5 minutes and then with magnesium stearate for 1 minute. The mixture thus obtained is compressed in a tablet press to produce tablets of suitable diameter.

### EXAMPLE 2d

### Tablets Containing 100 mg CGRP antagonist, 50 mg Sumatriptan and 10 mg Domperidone Maleate

Composition:

| | | |
|---|---|---|
| CGRP antagonist | 100 | mg |
| sumatriptan hydrogen | 70 | mg (corresponds to 50 mg sumatriptan) |
| succinate | | |
| domperidone maleate | 12.7 | mg (corresponds to 10 mg domperidone) |
| lactose | 303 | mg |
| microcrystalline | 112 | mg |
| cellulose | | |
| magnesium stearate | 9.3 | mg |
| Croscarmellose | 9.4 | mg |
| volatile constituent: | | |
| water | | |

Preparation

CGRP antagonist, sumatriptan hydrogen succinate, domperidone maleate, lactose (fine) and microcrystalline cellulose are homogeneously mixed in a suitable mixer (e.g. Diosna P2); then the mixture is granulated with water. The granulated material is screened through a 1.6 mm Kressner screen and dried for 2 hours at 40° C. Then the granules are screened in a suitable mill, e.g. a Comill, with a mesh size of 1.1 mm at 3000 rpm. The granules are then mixed with croscarmellose for 5 minutes and then with magnesium stearate for 1 minute. The mixture thus obtained is compressed in a tablet press to produce tablets of suitable diameter.

These methods form the basis for other examples of combinations listed in the Table that follows. In these Examples 10 to 600 mg CGRP antagonist was used, either in active form or in the form of a physiologically acceptable salt, combined with an amount, as listed in the Table, of an additional active substance or one of the physiologically acceptable salts thereof.

Table relating to Example 2a-d

| | | | | | | | | mg of | mg of | | |
|---|---|---|---|---|---|---|---|---|---|---|---|
| | A | | B1 | | B2 | | mg of | microcryst. | Mg- | mg of | Ø |
| Ex. | no. | mg | no. | mg | no. | mg | lactose | cellulose | stearate | croscarmellose | [mm] |
| 2.1 | 5 | 210 | 28 | 50 | | | 420.0 | 140.0 | 12.6 | 12.8 | 11 |
| 2.2 | 4a | 190 | 40 | 20 | | | 321.3 | 107.1 | 9.6 | 9.8 | 11 |
| 2.3 | 6 | 160 | 60 | 20 | 25 | 25 | 313.8 | 104.6 | 9.4 | 9.6 | 7 |
| 2.4 | 6a | 80 | 51 | 2.5 | | | 123.8 | 41.3 | 3.7 | 3.8 | 9 |
| 2.5 | 6 | 60 | 27 | 60 | | | 180.0 | 60.0 | 5.4 | 5.5 | 11 |
| 2.6 | 3 | 330 | 29 | 50 | | | 570.0 | 190.0 | 17.1 | 17.4 | 11 |
| 2.7 | 2a | 30 | 29 | 50 | 44a | 10 | 139.1 | 46.4 | 4.2 | 4.2 | 7 |
| 2.8 | 2 | 600 | 30 | 7.5 | | | 341.3 | 113.8 | 10.2 | 10.4 | 9 |
| 2.9 | 4 | 70 | 37 | 80 | | | 225.0 | 75.0 | 6.8 | 6.9 | 12 |
| 2.10 | 2 | 40 | 40 | 10 | | | 75.0 | 25.0 | 2.3 | 2.3 | 9 |
| 2.11 | 1 | 20 | 41 | 20 | | | 60.0 | 20.0 | 1.8 | 1.8 | 10 |
| 2.12 | 5a | 290 | 41 | 20 | 62 | 2.5 | 469.2 | 156.4 | 14.1 | 14.3 | 6 |
| 2.13 | 6 | 270 | 44 | 10 | | | 424.1 | 141.4 | 12.7 | 12.9 | 10 |
| 2.14 | 1 | 230 | 45a | 25 | | | 382.5 | 127.5 | 11.5 | 11.6 | 10 |
| 2.15 | 1a | 30 | 45a | 25 | 23 | 5 | 93.5 | 31.2 | 2.8 | 2.8 | 6 |
| 2.16 | 5 | 10 | 51 | 50 | | | 90.0 | 30.0 | 2.7 | 2.7 | 11 |
| 2.17 | 7 | 260 | 54 | 200 | | | 690.0 | 230.0 | 20.7 | 21.0 | 13 |
| 2.18 | 3 | 390 | 57 | 22 | | | 622.5 | 207.5 | 18.7 | 19.0 | 10 |
| 2.19 | 7 | 400 | 57 | 22 | 27 | 10 | 652.5 | 217.5 | 19.6 | 19.9 | 6 |
| 2.20 | 6 | 60 | 58 | 60 | 27 | 10 | 195.0 | 65.0 | 5.9 | 5.9 | 7 |

### EXAMPLE 3a

### Aqueous Solution for Intranasal Application Containing 20% CGRP Antagonist and 1 0% Sumatriptan

Composition:

| | | |
|---|---|---|
| CGRP antagonist | 20 | mg |
| sumatriptan | 10 | mg |
| mannitol | 5 | mg |
| water | ad 0.1 | ml |

Method

The two active substances are dissolved/suspended in water with stirring and optionally heating. The isotonic agent mannitol is added and the solution is made up to the final volume with water.

### EXAMPLE 3b

### Aqueous Solution for Intranasal Application Containing 2% CGRP Antagonist and 10% Sumatriptan

Composition:

| | | |
|---|---|---|
| CGRP antagonist | 2 | mg |
| sumatriptan | 10 | mg |
| mannitol | 5 | mg |
| water | ad 0.1 | ml |

Method

The two active substances are dissolved/suspended in water with stirring and optionally heating. The isotonic agent mannitol is added and the solution is made up to the final volume with water.

### EXAMPLE 3c

### Aqueous Solution for Intranasal Application Containing 40% CGRP Antagonist and 10% Sumatriptan

Composition:

| | | |
|---|---|---|
| CGRP antagonist | 40 | mg |
| sumatriptan | 10 | mg |
| mannitol | 5 | mg |
| water | ad 0.1 | ml |

ethod

The two active substances are dissolved/suspended in water with stirring and optionally heating. The isotonic agent mannitol is added and the solution is made up to the final volume with water.

### EXAMPLE 3d

### Aqueous Solution for Intranasal Application Containing 20% CGRP Antagonist, 2% Rizatriptan and 1.5% Labrasol

Composition:

| | | |
|---|---|---|
| CGRP antagonist | 20 | mg |
| rizatriptan | 2 | mg |
| labrasol | 1.5 | mg |
| mannitol | 5 | mg |
| water | ad 0.1 | ml |

Method

The two active substances are dissolved/suspended in water with stirring and optionally heating. The isotonic agent mannitol and labrasol are added and the solution is made up to the final volume with water.

### EXAMPLE 3e

### Aqueous Solution for Intranasal Application Containing 50% CGRP Antagonist, 2% Rizatriptan and 1.5% Labrasol

Composition:

| | | |
|---|---|---|
| CGRP antagonist | 50 | mg |
| rizatriptan | 2 | mg |
| labrasol | 1.5 | mg |
| mannitol | 5 | mg |
| water | ad 0.1 | ml |

Method

The two active substances are dissolved/suspended in water with stirring and optionally heating. The isotonic agent mannitol and labrasol are added and the solution is made up to the final volume with water.

These methods form the basis for other examples of combinations listed in the Table that follows.

In the Examples 2 to 50 mg CGRP antagonist was used, either in active form or in the form of a physiologically acceptable salt, combined with an amount, as listed in the Table, of an additional active substance or one of the physiologically acceptable salts thereof.

Table relating to Example 3a-e

| | substance | | substance | | mg of | mg of |
|---|---|---|---|---|---|---|
| Example | A no. | mg | B no. | mg | mannitol | labrasol |
| 3.1 | 3 | 10 | 29a | 3.5 | 5 | 3.00 |
| 3.2 | 3a | 20 | 56a | 4.8 | 5 | 3.00 |
| 3.3 | 3 | 15 | 31 | 2.5 | 5 | 1.50 |
| 3.4 | 4 | 20 | 45a | 2.8 | 5 | |
| 3.5 | 6 | 45 | 34 | 5.0 | 5 | |
| 3.6 | 2 | 20 | 25 | 2.0 | 5 | 3.00 |
| 3.7 | 5a | 40 | 37 | 6.0 | 5 | 1.50 |
| 3.8 | 4 | 20 | 28 | 5.0 | 5 | 1.50 |
| 3.9 | 2 | 5 | 27 | 4.0 | 5 | |
| 3.10 | 1 | 20 | 28 | 8.0 | 5 | |
| 3.11 | 1a | 50 | 64 | 4.0 | 5 | 3.00 |
| 3.12 | 3 | 2 | 39 | 2.5 | 5 | 3.00 |
| 3.13 | 4 | 20 | 42 | 4.0 | 5 | 3.00 |
| 3.14 | 4a | 35 | 44 | 2.0 | 5 | 3.00 |
| 3.15 | 2 | 20 | 70 | 5.0 | 5 | 1.50 |

Pellets

The medicament combinations according to the invention may also be prepared in the form of small particles such as e.g. pellets. The two active substances may be applied to neutral pellets consisting of sucrose and starch or microcrystalline cellulose, or separate pellets may be prepared for each active substance. These are then mixed together in the desired dosages in a capsule. A combination of different pellets is particularly advantageous if the active substances have to be administered in different dosages in order to achieve the optimum effect in the patient: If active substance A is administered in two doses and active substance B in three doses, this results in six fixed drug combinations, which require six different developments in the case of tablets, whereas in the case of pellets all that is needed is to mix different quantities of pellets and pack them into capsules.

If acidic or basic excipients make it easier for an active substance to dissolve, on account of the active substance having a pH-dependent solubility, it is also possible to use acidic or basic starter cores instead of neutral pellets.

If in the case of partial components there is a desire to prolong the duration of the effect, one or more types of pellet containing an active substance may also be coated with retarding lacquers. To do this, either pH-independently releasing lacquers such as e.g. ethylcellulose may be used with plasticisers/pore-forming agents such as polyethyleneglycol and talc as lubricant or polyacrylic resins based on copolymers of methacrylic acid and methacrylic acid esters with the brand name Eudragit may be used, which then exhibit a pH-dependent release.

The preparation comprises the following steps:

1. selection or preparation of starter pellets

2. formation of the layer of active substance

Optional: coating pellets to improve their stability or correct the flavour or—if desired—delay the release of one or more active substances.

### EXAMPLE 4a

### Preparation of Basic Starter Cores

Composition:

| | |
|---|---|
| Povidone K25 | 3 parts by weight |
| Microcryst. cellulose | 20 parts by weight |
| Meglumin | 77 parts by weight |

77 parts by weight meglumin, 20 parts by weight microcryst. cellulose and 3 parts by weight Povidone K25 are mixed for 15 minutes in a gyro wheel mixer. Then the powder mixture is placed in a twin-screw extruder at a rate of approx. 1 kg/h together with water which is added using a metering pump. The metering of the water is automatically regulated so as to obtain a rated torque of approx. 19% in the extruder. The extrusion is carried out through a nozzle plate drilled with holes 0.8 mm in diameter.

The extruded strips are rounded off into pellets in a spheronizer, the rounding operation lasting for approx. 3 minutes at approx. 850 RPM.

The pellets are dried at 80° C. for approx. 1.5 hours in a fluidised bed dryer.

The core material is fractionated through a tumbler screening machine with different perforated bases with nominal mesh sizes of 0.71 to 1.25 mm. The fractions of material of between 0.71 and 0.90 and 0.90 and 1.12 mm which are appropriate in each case are used in the later processes.

### EXAMPLE 4b

### Preparation of an Application of Active Substance Containing 100 Parts by Weight of CGRP Antagonist and 70 mg of Sumatriptan

Composition:

| | |
|---|---|
| core material | 200 parts by weight |
| hydroxypropylcellulose | 38 parts by weight |
| talc | 20 parts by weight |
| CGRP antagonist | 100 parts by weight |
| sumatriptan hydrogen succinate | 70 parts by weight |

Hydroxypropylcellulose is dissolved with stirring in 250 parts by weight of 2-propanol and then the active substances and talc are dispersed in this solution with stirring. In a fluidised bed processing apparatus 200 parts by weight of core material are sprayed with the dispersion containing the active substance at an air entry temperature of 20° to 30° C. by the under-bed spraying method. The pellets containing the active substance are then dried in the circulating air dryer at 35° C. for 8 hours.

To remove lumps, the pellets containing the active substance are screened using a screen with a nominal mesh size of 1.25 mm. The fraction of material (particle size <1.25 mm) is processed further.

The active substance layer is generally built up in the same way every time, but the type and amount of active substance, the nature and quantity of the binder, the amount of talc and the amounts of water, isopropanol or ethanol vary.

### EXAMPLE 4c

### Preparation of an Application of Active Substance Containing 10 Parts by Weight of CGRP Antagonist and 70 mg Sumatriptan

Composition:

| | |
|---|---|
| core material | 100 parts by weight |
| hydroxypropylcellulose | 24 parts by weight |
| talc | 12 parts by weight |
| CGRP antagonist | 10 parts by weight |
| sumatriptan hydrogen succinate | 70 parts by weight |

Hydroxypropylcellulose is dissolved with stirring in 250 parts by weight of 2-propanol and then the active substances and talc are dispersed in this solution with stirring. In a fluidised bed processing apparatus 100 parts by weight of core material are sprayed with the dispersion containing the active substance at an air entry temperature of 20° to 30° C. by the under-bed spraying method. The pellets containing the active substance are then dried in the circulating air dryer at 35° C. for 8 hours.

To remove lumps, the pellets containing the active substance are screened using a screen with a nominal mesh size of 1.25 mm. The fraction of material (particle size <1.25 mm) is processed further.

The active substance layer is generally built up in the same way every time, but the type and amount of active substance, the nature and quantity of the binder, the amount of talc and the amounts of water, isopropanol or ethanol vary.

### EXAMPLE 4d

### Preparation of an Application of Active Substance Containing 400 Parts by Weight of CGRP Antagonist and 70 mg Sumatriptan

Composition:

| | |
|---|---|
| core material | 100 parts by weight |
| hydroxypropylcellulose | 62 parts by weight |
| talc | 24 parts by weight |
| CGRP antagonist | 400 parts by weight |
| sumatriptan hydrogen succinate | 70 parts by weight |

Hydroxypropylcellulose is dissolved with stirring in 250 parts by weight of 2-propanol and then the active substances and talc are dispersed in this solution with stirring. In a fluidised bed processing apparatus 100 parts by weight of core material are sprayed with the dispersion containing the active substance at an air entry temperature of 20° to 30° C. by the under-bed spraying method. The pellets containing the active substance are then dried in the circulating air dryer at 35° C. for 8 hours.

To remove lumps, the pellets containing the active substance are screened using a screen with a nominal mesh size of 1.25 mm. The fraction of material (particle size <1.25 mm) is processed further.

The active substance layer is generally built up in the same way every time, but the type and amount of active substance, the nature and quantity of the binder, the amount of talc and the amounts of water, isopropanol and ethanol vary.

The respective compositions vary for each active substance combination and are shown in tabulated form hereinafter.

The Examples contain 10 to 600 parts by weight of CGRP antagonist either as an active form or in the form of a physiologically acceptable salt, while the rest of the composition is shown in the following Table.

Table relating to Example 4b-d

| | A | | B | | pbw | pbw | pbw | pbw | pbw | pbw | pbw |
|---|---|---|---|---|---|---|---|---|---|---|---|
| Ex. | no. | pbw | no. | pbw | povidone | HPC | pellets | talc | isopropanol | ethanol | water |
| 4.1 | 21 | 180 | 30a | 24.2 | 28.6 | 0.0 | 143.2 | 31.5 | 1890 | 0 | 0 |
| 4.2 | 2a | 220 | 41a | 2.5 | 24.3 | 0.0 | 121.5 | 26.7 | 1600 | 0 | 1600 |
| 4.3 | 7 | 150 | 32a | 2.8 | 24.4 | 0.0 | 121.8 | 26.8 | 0 | 1610 | 0 |
| 4.4 | 16 | 200 | 53a | 14.5 | 0.0 | 26.7 | 133.5 | 29.4 | 0 | 0 | 1760 |
| 4.5 | 5a | 260 | 64 | 2.5 | 0.0 | 24.3 | 121.5 | 26.7 | 0 | 1600 | 0 |
| 4.6 | 4 | 20 | 24 | 5.0 | 0.0 | 24.8 | 124.0 | 27.3 | 0 | 1640 | 0 |
| 4.7 | 1a | 50 | 35 | 10.0 | 25.8 | 0.0 | 129.0 | 28.4 | 1700 | 0 | 0 |
| 4.8 | 2 | 390 | 45 | 80.0 | 39.8 | 0.0 | 199.0 | 43.8 | 2630 | 0 | 0 |
| 4.9 | 6a | 10 | 26 | 100.0 | 0.0 | 43.8 | 219.0 | 48.2 | 0 | 2890 | 0 |
| 4.10 | 6 | 360 | 29 | 25.0 | 28.8 | 0.0 | 144.0 | 31.7 | 1900 | 0 | 0 |
| 4.11 | 12 | 120 | 69 | 50.0 | 33.8 | 0.0 | 169.0 | 37.2 | 2230 | 0 | 0 |
| 4.12 | 4a | 30 | 70 | 100.0 | 0.0 | 43.8 | 219.0 | 48.2 | 0 | 0 | 2890 |
| 4.13 | 21 | 50 | 64 | 200.0 | 0.0 | 63.8 | 319.0 | 70.2 | 4210 | 0 | 0 |
| 4.14 | 6 | 240 | 25 | 25.0 | 0.0 | 28.8 | 144.0 | 31.7 | 1900 | 0 | 0 |
| 4.15 | 17 | 340 | 25 | 50.0 | 0.0 | 33.8 | 169.0 | 37.2 | 2230 | 0 | 0 |
| 4.16 | 4 | 340 | 30 | 15.0 | 0.0 | 26.8 | 134.0 | 29.5 | 0 | 1800 | 0 |
| 4.17 | 14 | 110 | 31 | 250.0 | 0.0 | 73.8 | 369.0 | 81.2 | 0 | 0 | 4950 |
| 4.18 | 7a | 320 | 36 | 75.0 | 38.8 | 0.0 | 194.0 | 42.7 | 2600 | 0 | 0 |
| 4.19 | 4 | 160 | 36 | 150.0 | 53.8 | 0.0 | 269.0 | 59.2 | 3610 | 0 | 0 |
| 4.20 | 4a | 600 | 37 | 80.0 | 0.0 | 39.8 | 199.0 | 43.8 | 0 | 0 | 2670 |
| 4.21 | 3 | 350 | 41 | 20.0 | 0.0 | 27.8 | 139.0 | 30.6 | 1870 | 0 | 0 |
| 4.22 | 22 | 170 | 41 | 80.0 | 0.0 | 39.8 | 199.0 | 43.8 | 2670 | 0 | 0 |
| 4.23 | 1a | 30 | 44 | 12.7 | 0.0 | 26.3 | 131.7 | 29.0 | 0 | 0 | 1770 |
| 4.24 | 5 | 260 | 46a | 25.0 | 28.8 | 0.0 | 144.0 | 31.7 | 0 | 0 | 1930 |
| 4.25 | 14 | 190 | 48a | 5.9 | 25.0 | 0.0 | 124.9 | 27.5 | 0 | 1680 | 0 |
| 4.26 | 3 | 240 | 50 | 60.0 | 35.8 | 0.0 | 179.0 | 39.4 | 0 | 2400 | 0 |
| 4.27 | 3a | 200 | 50 | 120.0 | 47.8 | 0.0 | 239.0 | 52.6 | 0 | 3210 | 0 |
| 4.28 | 14 | 400 | 51 | 25.0 | 0.0 | 28.8 | 144.0 | 31.7 | 0 | 0 | 1930 |

*pbw = parts by weight;
HPC = hydroxypropylcellulose

Because of the properties of the active substances Examples 4.17, 4.18, 4.22 and 4.23 contain basic starter pellets instead of the neutral starter pellets.

### EXAMPLE 4e

### Isolation of the Active Substance-containing Pellets

Composition:

| | |
|---|---|
| Pellets containing active substance | 23 parts by weight |
| Gum arabic | 1 part by weight |
| Talc | 2 parts by weight |

1 part by weight of gum arabic is dissolved with stirring in a mixture of 6.7 parts by weight of ethanol 96% and 13.5 parts by weight of purified water. Then 2 parts by weight of talc are dispersed in the solution with stirring.

In a fluidised bed processing apparatus 23 parts by weight of pellets containing active substance are sprayed with the gum arabic/talc dispersion at an air entry temperature of 35° C. to 40° C. by the under-bed spraying method. The isolated core material is then dried in the circulating air dryer at 40° C. for 8 hours.

To remove lumps, the dried pellets containing active substance are screened using a screen with a nominal mesh size of 1.5 mm. The fraction of material (particle size <1.5 mm) is processed further.

### EXAMPLE 4f

### Delaying the Release of the Pellets Containing the Active Substance

Composition:

| | |
|---|---|
| pellets containing active substance | 30 parts by weight |
| Eudragit S 100 | 4 parts by weight |
| Eudragit RS 100 | 2 parts by weight |
| triethylcitrate | 1.25 parts by weight |
| hydroxypropylcellulose | 0.61 parts by weight |
| talc | 0.25 parts by weight |

4 parts by weight Eudragit S100, 2 parts by weight Eudragit RS100, 1.25 parts by weight triethylcitrate and 0.61 parts by weight hydroxypropylcellulose are dissolved In 112 parts by weight of 96% ethanol with stirring. Then 0.25 parts by weight of talc are dispersed in the solution with stirring.

In a fluidised bed processing apparatus 30 parts by weight of pellets containing active substance are sprayed with the delayed-release dispersion at an air entry temperature of 35° C. to 40° C. by the under-bed spraying method.

The isolated core material is then dried in the circulating air dryer at 40° C. for 8 hours. To remove lumps, the dried delayed-release pellets are screened using a screen with a nominal mesh size of 1.5 mm. The fraction of material (particle size <1.5 mm) is processed further.

A summary of the various delayed-release coatings is given in Table 4f.

TABLE 4F
The numbers correspond to parts by weight

Example

| | 4.29 | 4.30 | 4.31 | 4.32 |
|---|---|---|---|---|
| pellets of active | 30 | 30 | 30 | 30 |
| substance | | | | |
| ethylcellulose | 4 | 6 | | |
| polyethyleneglycol | 0.5 | 0.4 | | |
| Eudragit S100 | | | 3 | 5 |
| Eudragit RS100 | | | 3 | 1 |
| triethylcitrate | | | 1.25 | 1.25 |
| hydroxypropylcellulose | | | 0.61 | 0.61 |
| talc | 1.2 | 1.0 | 0.25 | 0.25 |

### EXTRUDATES

### Extruded Materials

The drug combinations according to the invention may also be prepared in the form of extruded materials which after being cut up or spheronised are packed directly into capsules or ground up and then made into tablets. The two active substances may be extruded together, or separate extrudate may be prepared for each active substance, and these are then mixed in a capsule in the desired dosages. A combination of different extruded materials is particularly advantageous if the active substances have to be administered in different dosages in order to achieve the optimum effect in the patient: If active substance A is administered in two doses and active substance B in three doses, this results in six fixed drug combinations, which require six different developments in the case of tablets, whereas in the case of extruded materials all that is needed is to mix different quantities of extruded materials and pack them into capsules.

The preparation comprises the following steps:
1. Extrusion
2a. Cutting up/spheronising
2b. Grinding and then processing to form tablets

### EXAMPLE 5a

### Preparation of Wet Extruded Materials

Composition:

| | |
|---|---|
| Povidone K25 | 6 parts by weight |
| microcrystalline cellulose | 40 parts by weight |
| CGRP antagonist | 100 parts by weight |
| sumatriptan hydrogen succinate | 70 parts by weight |

100 parts by weight of CGRP antagonist, 70 parts by weight of sumatriptan hydrogen succinate, 40 parts by weight microcrystalline cellulose (Avicel PH 101) and 6 parts by weight of povidone (Collidone K25) are mixed for 15 minutes in a gyro wheel mixer. Then the powder mixture is placed in a twin-screw extruder at a rate of approx. 1 kg/h together with water which is added using a metering pump. The metering of the water is automatically regulated so as to obtain a rated torque of approx. 19% in the extruder. The extrusion is carried out through a nozzle plate drilled with holes 0.8 mm in diameter.

The extruded strips are rounded off into pellets in a spheronizer, the rounding operation lasting for approx. 3 minutes at about 850 rpm.

The pellets are dried at 80° C. for approx. 1.5 hours in a fluidised bed dryer.

The core material is fractionated through a tumbler screening machine with different perforated bases with nominal mesh sizes of 0.71 to 1.25 mm. The fractions of material of between 0.71 and 0.90 and 0.90 and 1.12 mm which are appropriate in each case are used in the later processes.

### EXAMPLE 5b

### Preparation of Wet Extruded Material

Composition:

| | |
|---|---|
| povidone K25 | 4 parts by weight |
| microcrystalline cellulose | 30 parts by weight |
| CGRP antagonist | 10 parts by weight |
| sumatriptan hydrogen succinate | 70 parts by weight |

100 parts by weight of CGRP antagonist, 70 parts by weight of sumatriptan hydrogen succinate, 30 parts by weight microcrystalline cellulose (Avicel PH 101) and 4 parts by weight of povidone (Collidone K25) are mixed for 15 minutes in a gyrowheel mixer. Then the powder mixture is placed in a twin-screw extruder at a rate of approx. 1 kg/h together with water which is added using a metering pump. The metering of the water is automatically regulated so as to obtain a rated torque of approx. 19% in the extruder. The extrusion is carried out through a nozzle plate drilled with holes 0.8 mm in diameter.

The extruded strips are rounded off into pellets in a spheronizer, the rounding operation lasting for approx. 3 minutes at about 850 rpm.

The pellets are dried at 80° C. for approx. 1.5 hours in a fluidised bed dryer.

The core material is fractionated through a tumbler screening machine with different perforated bases with nominal mesh sizes of 0.71 to 1.25 mm. The fractions of material of between 0.71 and 0.90 and 0.90 and 1.12 mm which are appropriate in each case are used in the later processes.

### EXAMPLE 5c

### Preparation of Wet Extruded Material

Composition:

| | |
|---|---|
| povidone K25 | 15 parts by weight |
| microcrystalline cellulose | 110 parts by weight |
| CGRP antagonist | 400 parts by weight |
| sumatriptan hydrogen succinate | 70 parts by weight |

400 parts by weight of CGRP antagonist, 70 parts by weight sumatriptan hydrogen succinate, 110 parts by weight microcrystalline cellulose (Avicel PH 101) and 15 parts by weight of povidone (Collidone K25) are mixed for 15 minutes in a gyrowheel mixer. Then the powder mixture is placed in a twin-screw extruder at a rate of approx. 1 kg/h together with water which is added using a metering pump. The metering of the water is automatically regulated so as to obtain a rated torque of approx. 19% in the extruder. The extrusion is carried out through a nozzle plate drilled with holes 0.8 mm in diameter.

The extruded strips are rounded off into pellets in a spheronizer, the rounding operation lasting for approx. 3 minutes at about 850 rpm.

The pellets are dried at 80° C. for approx. 1.5 hours in a fluidised bed dryer.

The core material is fractionated through a tumbler screening machine with different perforated bases with nominal mesh sizes of 0.71 to 1.25 mm. The fractions of material of between 0.71 and 0.90 and 0.90 and 1.12 mm which are appropriate in each case are used in the later processes.

This preparation method is the basis for further combined examples which are listed in the following Table.

Table relating to Example 5a-c

| | | | | | | *pbw | *pbw | |
|---|---|---|---|---|---|---|---|---|
| | subst. A | *pbw | subst. | *pbw | subst. B2 | subst. | microcryst. | *pbw |
| Ex. | no. | subst. A | B1 no. | subst. B1 | no. | B2 | cellulose | povidone |
| 10.1 | 6 | 20 | 44 | 10 | | | 6.5 | 1.0 |
| 10.2 | 11a | 370 | 45a | 25 | 23 | 5 | 80.5 | 12.1 |
| 10.3 | 4a | 160 | 60 | 20 | | | 36.8 | 5.5 |
| 10.4 | 6a | 110 | 71 | 2.5 | | | 22.5 | 3.4 |
| 10.5 | 15 | 110 | 51 | 50 | | | 32.0 | 4.8 |
| 10.6 | 6 | 370 | 40 | 20 | 25 | 25 | 83.8 | 12.6 |
| 10.7 | 5 | 250 | 28 | 50 | | | 64.0 | 9.6 |
| 10.8 | 2a | 370 | 39 | 50 | 44a | 10 | 86.5 | 13.0 |
| 10.9 | 7 | 390 | 57 | 22 | 27 | 10 | 85.0 | 12.8 |
| 10.10 | 17 | 40 | 54 | 200 | | | 48.0 | 7.2 |
| 10.11 | 22 | 200 | 40 | 10 | | | 42.0 | 6.3 |
| 10.12 | 2 | 30 | 30 | 7.5 | | | 7.5 | 1.1 |
| 10.13 | 1 | 380 | 41 | 20 | | | 80.0 | 12.0 |
| 10.14 | 15a | 360 | 41 | 20 | 32 | 2.5 | 76.6 | 11.5 |
| 10.15 | 1 | 250 | 45a | 25 | | | 55.0 | 8.3 |
| 10.16 | 3 | 160 | 57 | 22 | | | 37.0 | 5.6 |
| 10.17 | 14 | 10 | 37 | 80 | | | 18.0 | 2.7 |
| 10.18 | 6 | 380 | 58 | 60 | 27 | 10 | 90.0 | 13.5 |
| 10.19 | 16 | 160 | 27 | 60 | | | 44.0 | 6.6 |
| 10.20 | 3 | 260 | 29 | 50 | | | 62.0 | 9.3 |

*pbw = parts by weight

### EXAMPLE 6a

### Preparation of Molten Extruded Material

Composition:

| | |
|---|---|
| povidone K25 | 6 parts by weight |
| poloxamer | 40 parts by weight |
| CGRP antagonist | 100 parts by weight |
| sumatriptan hydrogen succinate | 70 parts by weight |

100 parts by weight of CGRP antagonist, 70 parts by weight sumatriptan hydrogen succinate, 40 parts by weight poloxamer and 6 parts by weight povidone K25 are mixed for 15 minutes in a gyrowheel mixer. Then the powder mixture is placed in a twin-screw extruder at a rate of approx. 1 kg/h together with water which is added using a metering pump. The temperature is regulated so as to obtain a rated torque of approx. 19% in the extruder. The extrusion is carried out through a nozzle plate drilled with holes 0.8 mm in diameter.

The extruded strips emerging are cut by chopping off the top, and the extruded strips are rounded off into pellets in a spheronizer, the rounding operation lasting for approx. 3 minutes at about 850 rpm.

The pellets are dried at 80° C. for approx. 1.5 hours in a fluidised bed dryer.

The core material is fractionated through a tumbler screening machine with different perforated bases with nominal mesh sizes of 0.71 to 1.25 mm. The fractions of material of between 0.71 and 0.90 and 0.90 and 1.12 mm which are appropriate in each case are used in the later processes.

### EXAMPLE 6b

### Preparation of Molten Extruded Material

Composition:

| | |
|---|---|
| povidone K25 | 2 parts by weight |
| poloxamer | 30 parts by weight |
| CGRP antagonist | 10 parts by weight |
| sumatriptan hydrogen succinate | 70 parts by weight |

10 parts by weight of CGRP antagonist, 70 parts by weight sumatriptan hydrogen succinate, 30 parts by weight poloxamer and 2 parts by weight povidone K25 are mixed for 15 minutes in a gyrowheel mixer. Then the powder mixture is placed in a twin-screw extruder at a rate of approx. 1 kg/h together with water which is added using a metering pump. The temperature is regulated so as to obtain a rated torque of approx. 19% in the extruder. The extrusion is carried out through a nozzle plate drilled with holes 0.8 mm in diameter.

The extruded strips emerging are cut by chopping off the top, and the extruded strips are rounded off into pellets in a spheronizer, the rounding operation lasting for approx. 3 minutes at about 850 rpm at about 40° C.

The pellets are dried at 80° C. for approx. 1.5 hours in a fluidised bed dryer.

The core material is fractionated through a tumbler screening machine with different perforated bases with nominal mesh sizes of 0.71 to 1.25 mm. The fractions of material of between 0.71 and 0.90 and 0.90 and 1.12 mm which are appropriate in each case are used in the later processes.

### EXAMPLE 6c

### Preparation of Molten Extruded Material

Composition:

| | |
|---|---|
| povidone K25 | 18 parts by weight |
| poloxamer | 132 parts by weight |
| CGRP antagonist | 400 parts by weight |
| sumatriptan hydrogen succinate | 70 parts by weight |

400 parts by weight of CGRP antagonist, 70 parts by weight sumatriptan hydrogen succinate, 132 parts by weight poloxamer and 18 parts by weight povidone K25 are mixed for 15 minutes in a gyrowheel mixer. Then the powder mixture is placed in a twin-screw extruder at a rate of approx. 1 kg/h together with water which is added using a metering pump. The temperature is regulated so as to obtain a rated torque of approx. 19% in the extruder. The extrusion is carried out through a nozzle plate drilled with holes 0.8 mm in diameter.

The extruded strips emerging are cut by chopping off the top, and the extruded strips are rounded off into pellets in a spheronizer, the rounding operation lasting for approx. 3 minutes at about 850 rpm at about 40° C.

The pellets are dried at 80° C. for approx. 1.5 hours in a fluidised bed dryer.

The core material is fractionated through a tumbler screening machine with different perforated bases with nominal mesh sizes of 0.71 to 1.25 mm. The fractions of material of between 0.71 and 0.90 and 0.90 and 1.12 mm which are appropriate in each case are used in the later processes.

The different compositions may vary, and further Examples are given below in the form of a Table.

Table relating to Example 6a-c

| | subst. | pbw | subst. | pbw | subst. | pbw | pbw | pbw |
|---|---|---|---|---|---|---|---|---|
| Ex. | A no. | subst A | B1 no. | subst. B1 | B2 no. | subst. B2 | povidone | poloxamer |
| 6.1 | 7a | 140 | 58 | 60 | | | 3.0 | 50.8 |
| 6.2 | 7 | 200 | 57 | 22.1 | | | 3.4 | 57.1 |
| 6.3 | 12a | 330 | 53 | 25 | | | 5.4 | 90.7 |
| 6.4 | 3a | 160 | 30 | 15 | | | 2.6 | 44.4 |
| 6.5 | 16a | 10 | 8a | 50 | 45 | 25 | 1.6 | 26.6 |
| 6.6 | 2 | 230 | 24 | 2.5 | | | 3.5 | 59.0 |
| 6.7 | 6 | 140 | 39 | 12.5 | | | 2.3 | 38.7 |
| 6.8 | 5a | 70 | 50 | 60 | | | 2.0 | 33.0 |
| 6.9 | 1a | 390 | 44a | 10 | 12a | 2.78 | 6.1 | 102.9 |
| 6.10 | 13 | 330 | 43a | 10 | | | 5.1 | 86.4 |
| 6.11 | 16 | 380 | 24 | 2.5 | 58 | 60 | 6.6 | 112.3 |
| 6.12 | 3 | 360 | 41 | 40 | | | 6.0 | 101.5 |
| 6.13 | 15 | 270 | 46a | 25 | | | 4.4 | 74.9 |
| 6.14 | 5 | 400 | 56 | 60 | | | 6.9 | 116.7 |
| 6.15 | 21 | 280 | 36 | 75 | | | 5.3 | 90.1 |
| 6.16 | 4a | 230 | 41 | 40 | 18 | 25 | 4.4 | 74.9 |
| 6.17 | 4 | 120 | 58 | 60 | 30 | 15 | 2.9 | 49.5 |
| 6.18 | 5 | 150 | 48 | 5 | | | 2.3 | 39.6 |
| 6.19 | 17 | 180 | 26 | 100 | | | 4.2 | 71.1 |
| 6.20 | 4 | 30 | 52 | 150 | | | 0.5 | 7.7 |
| 6.21 | 1a | 190 | 43 | 10 | | | 3.1 | 51.9 |
| 6.22 | 3 | 390 | 8a | 50 | | | 6.9 | 116.7 |
| 6.23 | 4 | 210 | 30 | 15 | 54 | 200 | 6.4 | 107.8 |
| 6.24 | 3 | 290 | 25 | 40 | | | 5.0 | 83.7 |
| 6.25 | 12 | 60 | 44a | 10 | | | 1.1 | 18.5 |

*pbw = parts by weight

### EXAMPLE 7

### Further Processing to Form Tablets

The extruded materials are ground up in a suitable mill and the resulting granulated material is further processed with conventional tabletting excipients analogously to Example 1 to produce tablets.

### POWDER INHALANT

### Preparation of Spherically Nanostructured Microparticles of the Active Substances for Preparing a Powder Inhalant

The active substances are dissolved in an ethanol/water (4:1) mixture in order to prepare a 4 wt.% active substance solution and the active substance solution is sprayed so as to produce a spray mist with a droplet size having the characteristic value X50 (median value=the particle size/droplet size below which 50% of the quantity of particles falls, with respect to the volume distribution of the individual particles/drops) in the range from 1.5 to 8 μm, and wherein Q(5.8) (corresponds to the amount of particles below 5.8 μm, based on the volume distribution of the droplets) is between 30% and 100%. The spray mist thus obtained is dried using a drying gas with an entry temperature of between 130° C. and 200° C. and an exit temperature of 40° C. to 120° C.. The flow volume of the spray gas is 1 Nm³/h to 15 Nm³/h and the flow volume of the drying gas is 15 Nm³/h to 150 Nm³/h. The dried solid fraction is collected using a gravity separator and/or filter unit.

### EXAMPLE 8

### Capsules for Powder Inhalation Containing 0.5 mg CGRP Antagonist and 0.35 mg Sumatriptan

Composition:
1 capsule for powder inhalation contains:

| | |
|---|---|
| CGRP antagonist | 0.5 mg |
| sumatriptan | 0.35 mg |
| lactose | 20 mg |
| hard gelatine capsules | 50 mg |

Preparation Method

The active substances are prepared as spherically nanostructured active substance particles and homogeneously mixed with lactose. The mixture is packed into hard gelatine capsules.

The particular compositions vary for each active substance combination and are shown in table form below.

The Examples contain 0.1 to 45 mg of CGRP antagonist, the remainder of the composition is shown in the following Table.

Table relating to Example 8

| Example | substance A | | | | |
|---|---|---|---|---|---|
| no. | no. | mg | substance B no. | mg | mg lactose |
| 8.1 | 4 | 0.70 | 29 | 0.12 | 49.00 |
| 8.2 | 12 | 5.00 | 30 | 1.21 | 42.80 |
| 8.3 | 21 | 45.00 | 41 | 1.13 | 5.30 |
| 8.4 | 6 | 4.00 | 32 | 0.11 | 45.10 |
| 8.5 | 16 | 3.00 | 23 | 0.22 | 46.20 |
| 8.6 | 1 | 3.00 | 24 | 0.15 | 46.30 |
| 8.7 | 3 | 6.00 | 65 | 0.60 | 42.30 |
| 8.8 | 1 | 30.00 | 67 | 9.00 | 5.30 |
| 8.9 | 3 | 7.00 | 58 | 1.75 | 39.90 |
| 8.10 | 1 | 5.00 | 29 | 1.25 | 42.80 |
| 8.11 | 15 | 20.00 | 25 | 5.00 | 21.20 |
| 8.12 | 5 | 30.00 | 27 | 3.00 | 11.30 |
| 8.13 | 16 | 45.00 | 28 | 3.60 | 2.90 |
| 8.14 | 2 | 10.00 | 30 | 0.75 | 37.40 |
| 8.15 | 22 | 16.00 | 34 | 1.60 | 29.40 |
| 8.16 | 5 | 20.00 | 39 | 2.50 | 23.70 |
| 8.17 | 16 | 10.00 | 41 | 2.00 | 36.10 |
| 8.18 | 2 | 40.00 | 42 | 1.60 | 0.80 |
| 8.19 | 4 | 30.00 | 43a | 3.16 | 11.10 |
| 8.20 | 14 | 5.00 | 44 | 0.64 | 43.40 |
| 8.21 | 6 | 10.00 | 45 | 2.50 | 35.60 |
| 8.22 | 13 | 5.00 | 46a | 1.25 | 42.80 |
| 8.23 | 4 | 30.00 | 48 | 1.77 | 12.50 |
| 8.24 | 4 | 25.00 | 51 | 6.25 | 14.00 |

### EXAMPLE 9

### Injectable Solution Containing 0.5 mg CGRP Antagonist and 0.35 mg Sumatrinptan

Composition:

| | |
|---|---|
| CGRP antagonist | 0.5 mg |
| sumatriptan | 35 mg |
| physiological saline solution | |

The active substances are dissolved in physiological saline solution.

The particular compositions vary for each active substance combination and are shown in table form below.

The Examples contain 0.2 to 30 mg of CGRP antagonist, the remainder of the composition is shown in the following Table.

Table relating to Example 9

| | CGRP | | substance B | |
|---|---|---|---|---|
| Example | antagonist no. | mg | no. | mg |
| 9.1 | 15 | 0.20 | 28 | 5.00 |
| 9.2 | 4a | 14.30 | 30 | 2.00 |
| 9.3 | 6 | 4.40 | 67 | 2.00 |
| 9.4 | 16a | 10.30 | 58 | 2.50 |
| 9.5 | 6 | 1.80 | 27 | 6.00 |
| 9.6 | 3 | 1.30 | 39 | 5.00 |
| 9.7 | 2a | 4.40 | 39 | 5.00 |
| 9.8 | 12 | 9.40 | 30 | 7.50 |
| 9.9 | 4 | 2.60 | 37 | 8.00 |
| 9.10 | 22 | 8.20 | 40 | 1.00 |
| 9.11 | 1 | 4.30 | 41 | 2.00 |
| 9.12 | 5a | 25.50 | 41 | 2.00 |
| 9.13 | 6 | 14.20 | 44 | 1.00 |
| 9.14 | 21 | 13.40 | 45a | 2.50 |
| 9.15 | 1a | 5.40 | 45a | 2.50 |
| 9.16 | 15 | 6.90 | 51 | 5.00 |
| 9.17 | 7 | 7.70 | 54 | 10.00 |
| 9.18 | 3 | 30.00 | 57 | 2.00 |
| 9.19 | 7 | 8.30 | 57 | 2.00 |
| 9.20 | 16 | 13.10 | 58 | 6.00 |

### EXAMPLE 10

### Suppositories Containing 200 mg CGRP Antagonist and 50 mg Sumatriptan

Composition:

| | | |
|---|---|---|
| CGRP antagonist | 200 | mg |
| sumatriptan hydrogen | 70 | mg (corresponds to 50 mg sumatriptan) |
| succinate | | |
| hard wax | ad 2 | g |

Preparation

The hard wax is melted and the active substances are suspended in the mass. Then the mass is poured into suitable suppository moulds.

The particular compositions vary for each active substance combination and are shown in table form below.

The Examples contain 50 to 600 mg of CGRP antagonist.

Table relating to Example 10

| | CGRP | | substance B | |
|---|---|---|---|---|
| Example | antagonist no. | mg | no. | mg |
| 1.1 | 13 | 250 | 28 | 100 |
| 1.2 | 6a | 150 | 28 | 100 |
| 1.3 | 1a | 460 | 43a | 20 |
| 1.4 | 22 | 540 | 34 | 5 |
| 1.5 | 6 | 320 | 35 | 5 |
| 1.6 | 13 | 180 | 45 | 80 |
| 1.7 | 7 | 150 | 56 | 200 |
| 1.8 | 3a | 480 | 30 | 30 |
| 1.9 | 4 | 600 | 30 | 30 |
| 1.10 | 5 | 180 | 48 | 10 |
| 1.11 | 11 | 520 | 36 | 150 |
| 1.12 | 6 | 540 | 39 | 25 |
| 1.13 | 3 | 110 | 41 | 80 |
| 1.14 | 4a | 560 | 41 | 80 |
| 1.15 | 3 | 50 | 43a | 20 |
| 1.16 | 12 | 320 | 44a | 20 |
| 1.17 | 1a | 440 | 44a | 20 |
| 1.18 | 5 | 590 | 46a | 50 |

1. A method for treating headache, migraine headache and cluster headache, comprising the joint administration of a therapeutically effective amount of a CGRP antagonists (A), which is selected from the group consisting of
(1) 4-(2-oxo-1,2,4,5-tetrahydro-1,3-benzodiazepin-3-yl)-piperidine-1-carboxylic acid-{(R)-1-(4-amino-3-chloro-5-ethyl-benzyl)-2-[4-(4-methyl-piperazin-1-yl) -piperidin-1-yl]-2-oxo-ethyl}-amide,
(2) [1′-((R)-3-(4-amino-3-chloro-5-trifluoromethyl-phenyl)-2-{[4-(2-oxo-1,2,4,5-tetra-hydro-1,3-benzodiazepin-3-yl) -piperidine-1-carbonyl]-amino}-propionyl)-4,4′-bi-piperidinyl-1-yl]-acetic acid,
(3) 3-{1-[(R)-1-(4-amino-3,5-dibromo-benzyl)-2-[1,4′]bipiperidinyl-1′-yl-2-oxo-ethyl-carbamoyl]-piperidin-4-yl}-2-oxo-1,2,3,4-tetrahydro-quinazoline-7-carboxylic acid,
(4) (R)-1-(7-methyl-1H-benzotriazol-5-ylmethyl)-2-[4-(4-methyl-piperazin-1-yl)-piperidin-1-yl]-2-oxo-ethyl 4-(2-oxo-1,2,4,5-tetrahydro-1,3-benzodiazepin-3-yl)-piperidine-1-carboxylate,
(5) (S)-2-(3-chloro-4-hydroxy-5-trifluoromethyl-benzyl)-1-[4-(1-methyl-piperidin-4-yl )-piperazin-1-yl]-4-[4-(2-oxo-1,2,4,5-tetrahydro-1,3-benzodiazepin-3-yl)-piperidin-1-yl]-butane-1,4-dione,
(6) (R)-1-(4-hydroxy-3,5-dimethyl-benzyl)-2-oxo-2-(4-piperidin-4-yl-piperazin-1-yl)-ethyl 4-(2-oxo-1,2,4,5-tetrahydro-1,3-benzodiazepin-3-yl)-piperidine-1-carboxylate,
(7) (R)-1-(3,5-dibromo-4-hydroxy-benzyl)-2-[4-(1-methyl-piperidin-4-yl)-piperazin-1-yl]-2-oxo-ethyl 4-(2-oxo-1,2,4,5-tetrahydro-1,3-benzodiazepin-3-yl)-piperidine-1-carboxylate,
(8) (R)-1-(6-amino-5-methyl-pyridin-3-ylmethyl)-2-oxo-2-(4-piperazin-1-yl-piperidin-1-yl)-ethyl 4-(2-oxo-1,2,4,5-tetrahydro-1,3-benzodiazepin-3-yl)-piperidine-1-carboxylate,
(9) (R)-1-(3,5-dibromo-4-hydroxy-benzyl)-2-oxo-2-(4-piperazin-1-yl-piperidin-1-yl)-ethyl 4-(2-oxo-1,2,4,5-tetrahydro-1,3-benzodiazepin-3-yl)-piperidine-1-carboxylate,
(10) (R)-1-(3,5-dibromo-4-hydroxy-benzyl )-2-oxo-2-(4-piperidin-4-yl-piperazin-1-yl )-ethyl 4-(2-oxo-1,2,4,5-tetrahydro-1,3-benzodiazepin-3-yl)-piperidine-1-carboxylate,
(11) (S)-2-(4-amino-3-chloro-5-trifluoromethyl-benzyl )-1-[4-(4-methyl-piperazin-1-yl)-piperidin-1-yl]-4-[4-(2-oxo-1,2,4,5-tetrahydro-1,3-benzodiazepin-3-yl)-piperidin-1-yl]-butan-1,4-dione,
(12) 4-(2-oxo-1,2,4,5-tetrahydro-1,3-benzodiazepin-3-yl)-piperidine-1 -carboxylic acid-{(R)-1-(3,4-diethyl-benzyl)-2-[4-(1-methyl-piperidin-4-yl)-piperazin-1-yl]-2-oxo-ethyl}-amide,
(13) (R)-1-(4-amino-3-chloro-5-trifluoromethyl-benzyl)-2-[4-(4-methyl-piperazin-1-yl)-piperidin-1-yl]-2-oxo-ethyl 4-(2-oxo-1,2,4,5-tetrahydro-1,3-benzodiazepin-3-yl)-piperidine-1-carboxylate,
(14) 4-(2-oxo-1,2,4,5-tetrahydro-1,3-benzodiazepin-3-yl)-piperidine-1-carboxylic acid-{(R)-1-(4-amino-3-chloro-5-trifluoromethyl-benzyl)-2-[4-(4-methyl-piperazin-1-yl) -piperidin-1-yl]-2-oxo-ethyl}-amide,
(15) (S)-2-(4-amino-3,5-bis-trifluoromethyl-benzyl)-1-[4-(4-methyl-piperazin-1-yl)-piperidin-1-yl]-4-[4-(2-oxo-1,2,4,5-tetrahydro-1,3-benzodiazepin-3-yl)-piperidin-1-yl]-butan-1,4-dione,
(16) 4-(2-oxo-1,2,4,5-tetrahydro-1,3-benzodiazepin-3-yl)-piperidine-1-carboxylic acid-{(R)-1-(4-amino-3,5-bis-trifluoromethyl-benzyl)-2-[4-(4-methyl-piperazin-1-yl) -piperidin-1-yl]-2-oxo-ethyl}-amide,
(17) (R)-1-(4-amino-3,5-bis-trifluoromethyl-benzyl)-2-[4-(4-methyl-piperazin-1-yl)-piperidin-1-yl]-2-oxo-ethyl 4-(2-oxo-1,2,4,5-tetrahydro-1,3-benzodiazepin-3-yl )-piperidine-1-carboxylate,
(18) 4-(2-oxo-1,2,4,5-tetrahydro-1,3-benzodiazepin-3-yl)-piperidine-1-carboxylic acid-{(R)-1-(4-amino-3-chloro-5-methyl-benzyl)-2-[4-(1-methyl-piperidin-4-yl)-piperazin-1-yl]-2-oxo-ethyl}-amide,
(19) (R)-1-(3,5-dibromo-4-hydroxy-benzyl)-2-[4-(4-methyl-piperazin-1-yl)-piperidin-1-yl]-2-oxo-ethyl 4-(2-oxo-1,2,4,5-tetrahydro-1,3-benzodiazepin-3-yl)-piperidine-1-carboxylate,
(20) (R)-1-(4-hydroxy-3,5-di methyl-benzyl)-2-oxo-2-(4-piperazin-1-yl-piperidin-1-yl)-ethyl 4-(2-oxo-1,2,4,5-tetrahydro-1,3-benzodiazepin-3-yl)-piperidine-1-carboxylate,
(21) (R)-1-(4-hydroxy-3,5-dimethyl-benzyl)-2-[4-(1-methyl-piperidin-4-yl)-piperazin-1-yl]-2-oxo-ethyl 4-(2-oxo-1,2,4,5-tetrahydro-1,3-benzodiazepin-3-yl)-piperidine-1-carboxylate, and
(22) (S)-1-1,4′-bipiperidinyl-1′-yl-2-(3-chloro-4-hydroxy-5-trifluoromethyl-benzyl)-4-[4-(2-oxo-1,2,4,5-tetrahydro-1,3-benzodiazepin-3-yl)-piperidin-1-yl]-butane-1,4-dione,
or a physiologically acceptable salt thereof, and a therapeutically effective amount of one or two other anti-migraine medicaments (B) to a person in need of such treatment.

2. The method according to claim 1, wherein the medicament (B) is selected from the group consisting of the angiotensin-II antagonists, α-agonists and α-antagonists, 5-HT_{1B/1D}-agonists, AMPA antagonists, antidepressants, antiemetics, anticonvulsants, antimuscarinics, β-blockers, calcium antagonists, corticosteroids, ergot alkaloids, histamine-H1-receptor antagonists, weak analgesics, neurokinin antagonists, neuroleptics, non-steroidal antiinflammatories, NO-synthase inhibitors, prokinetics and serotonin-reuptake inhibitors.

3. The method according to claim 2, wherein the medicament (B) is selected from among the ergot alkaloids and 5-HT_{1B/1D}-agonists.

4. The method according to claim 3, wherein the ergot alkaloid may be ergotamine or dihydroergotamine or a physiologically acceptable salt thereof and the the 5-HT_{1B/1D}-agonist may be almotriptan, avitriptan, donitriptan, eletriptan, frovatriptan, naratriptan, rizatriptan, sumatriptan or zolmitriptan or a physiologically acceptable salt thereof.

5. The method according to claim 4, wherein medicament (B) may be sumatriptan, zolmitriptan or dihydroergotamine or a physiologically acceptable salt thereof.

6. The method according to claim 5, wherein the selected CGRP antagonist (A), or a physiologically acceptable salt thereof is administered by intravenous or subcutaneous route in a dosage of 0.0001 to 3 mg/kg body weight, by oral route in a dosage of 01 to 20 mg/kg body weight or by nasal or inhalative route in a dosage of 0.1 to 10 mg/kg body weight once, twice or three times a day and
sumatriptan or a physiologically acceptable salt thereof is administered by oral route in a dosage of 0.03 to 1.43 mg/kg body weight once, twice or three times a day or
by intravenous or subcutaneous route in a dosage of 0.002 to 0.09 mg/kg body weight once or twice a day or
by rectal route in a dosage of 0.007 to 0.36 mg/kg body weight once or twice a day or
by nasal route in a dosage of 0.006 to 0.29 mg/kg body weight once or twice a day or
zolmitriptan or a physiologically acceptable salt thereof is administered by oral route in a dosage of 0.0007 to 0.036 mg/kg body weight once or twice a day or
dihydroergotamine or a physiologically acceptable salt thereof is administered by oral route in a dosage of 0.001 to 0.07 mg/kg body weight once or twice a day.

7. The method according to claim 2, wherein medicament (B) is a serotonin reuptake inhibitor.

8. The method according to claim 7, wherein the serotonin reuptake inhibitor is citalopram, duloxetine, escitalopram, fluoxetine, fluvoxamine, paroxetine, sertraline, or trazodone or a physiologically acceptable salt thereof.

9. The method according to claim 8, wherein medicament (B) is duloxetine or a physiologically acceptable salt thereof.

10. A pharmaceutical composition for the treatment or prevention of headache, migraine or cluster headache, comprising a therapeutically effective amount of a selected CGRP antagonist (A), a physiologically acceptable salt thereof or a hydrate of the salt and an anti-migraine medicament (B) which is selected from among sumatriptan, zolmitriptan and dihydroergotamine and a physiologically acceptable salt thereof, as a combined preparation for simultaneous or sequential administration.

11. A pharmaceutical composition according to claim 10, comprising a single dosage unit of 0.1 to 1500 mg of a selected CGRP-antagonist (A), a physiologically acceptable salt thereof or a hydrate of the salt and
a single dosage unit of 1 to 100 mg sumatriptan or
a single dosage unit of 0.1 to 2.5 mg zolmitriptan or
a single dosage unit of 0.1 to 5 mg dihydroergotamine.
